(19) United States
(12) Patent Application Publication
(10) Pub. No.: US 2023/0114445 A1
(43) Pub. Date: Apr. 13, 2023

(54) DEVICE FOR EX-SITU IMPLANT ORIENTATION AND ASSEMBLY

(71) Applicant: WishBone Medical, Inc., Warsaw, IN (US)

(72) Inventor: Reese Myers, Columbia City, IN (US)

(73) Assignee: WishBone Medical, Inc., Warsaw, IN (US)

(21) Appl. No.: 17/498,428

(22) Filed: Oct. 11, 2021

(51) Int. Cl. A61F 2/00; A61F 2/36

(52) U.S. Cl. CPC A61F 2/0095; A61F 2/3607; A61F 2230/0065; A61F 2002/30617

(57) ABSTRACT

A holding device for assembling orthopaedic implants includes: a stand including a columnar structure with an opening formed in the structure and configured to accept a stem and a groove formed in the structure that is configured to accept a portion of an orthopaedic implant to rotatably lock the orthopaedic implant in the opening and a base coupled to the structure and configured to stabilize the structure on a surface; and an angle set including a ring rotatably coupled to the structure and a fork configured to engage the orthopaedic implant and carried by the ring.

### BACKGROUND OF THE INVENTION

### 1. Field of the Invention

The present invention relates to orthopaedic implants, and, more particularly, to assemblies for preparing orthopaedic implants.

### 2. Description of the Related Art

Many orthopaedic implants are modular implants that include multiple, initially separated components. The components are typically assembled together in-situ, e.g., during a surgical procedure, to form the orthopaedic implant. While such modular orthopaedic implants provide surgeons with a variety of options to tailor the orthopaedic implant to the patient during surgery, modular implants are not without drawbacks.

For example, assembling the components together in-situ risks allowing bodily fluids and tissues to contaminate precision surfaces of the components during assembly, which can lead to an improper fit. Further, in-situ assembly does not always allow for proper orientation of forces, such as impacts from a mallet, during assembly. Off-axis impact forces can cause the assembled orthopaedic implant to have misaligned components, which can result in undesirable residual assembly stresses. Even further, a surgeon often utilizes a trial assembly in-situ to determine the desired orientation and fit of the final orthopaedic implant that will be implanted in the patient. Transferring the orientation of various components of the trial assembly to the components forming the final orthopaedic implant can be difficult and/or time-consuming, which can further complicate a surgical procedure and/or increase operating time and cost.

What is needed in the art is a holding device that can be conveniently used to form orthopaedic implants.

### SUMMARY OF THE INVENTION

The present invention provides a holding device with a groove and a fork that can engage portions of an orthopaedic implant to hold the orthopaedic implant for assembly.

The invention in one form is directed to a holding device for assembling orthopaedic implants, the device including: a stand including a columnar structure with an opening formed in the structure and configured to accept a stem and a groove formed in the structure that is configured to accept a portion of an orthopaedic implant to rotatably lock the orthopaedic implant in the opening and a base coupled to the structure and configured to stabilize the structure on a surface; and an angle set including a ring rotatably coupled to the structure and a fork configured to engage the orthopaedic implant and carried by the ring.

The invention in another form is directed to an orthopaedic implant construct including: an orthopaedic implant including a stem; and a holding device holding the orthopaedic implant. The holding device includes: a stand including a columnar structure with an opening formed in the structure and at least partially filled by the stem and a groove formed in the structure and at least partially filled by a portion of the orthopaedic implant so the orthopaedic implant is rotatably locked in the opening and a base coupled to the structure and configured to stabilize the structure on a surface; and an angle set including a ring rotatably coupled to the structure and a fork carried by the ring and engaging the held orthopaedic implant.

An advantage of the present invention is the holding device can be used to orient components of a modular orthopaedic implant before tightly coupling the components together.

Another advantage is the ring can be rotated about the structure to engage and reference a trial assembly.

Yet another advantage is the ring can be rotated to adjust an angle of a portion of the orthopaedic implant engaged by the fork.

Yet another advantage is the holding device can include angle indicators and an angle marker to identify an angle between the groove and the fork, which can be used to form an orthopaedic implant with a desired orientation between two or more components.

### BRIEF DESCRIPTION OF THE DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 is a perspective view of an exemplary embodiment of an orthopaedic implant construct provided according to the present invention including a holding device holding an orthopaedic implant that is partially assembled;

FIG. 2 is a perspective view of a portion of the holding device of FIG. 1 with the orthopaedic implant removed;

FIG. 3 is a perspective view of the orthopaedic implant construct of FIG. 1 with a fork moved to an engaging position to engage the orthopaedic implant so the orthopaedic implant can be fully assembled;

FIG. 4 is a top view of the orthopaedic implant construct of FIGS. 1 and 3;

FIG. 5 is a perspective view of another exemplary embodiment of a holding device provided according to the present invention;

FIG. 6 is a perspective view of a portion of the holding device of FIG. 5;

FIG. 7 is a perspective view of a trial assembly being inserted in the holding device of FIGS. 5-6;

FIG. 8 is a perspective view of the holding device of FIGS. 5-7 with the trial assembly of FIG. 7 fully inserted therein;

FIG. 9 is a perspective view of the holding device of FIGS. 5-8 holding a sleeve for an orthopaedic implant;

FIG. 10 is a perspective view of the holding device and the sleeve of FIG. 9 as well as an orthopaedic implant body prior to insertion in the sleeve and the holding device; and

FIG. 11 is a perspective view of an exemplary embodiment of an orthopaedic implant construct provided according to the present invention including the holding device and the sleeve of FIGS. 9-10 and the orthopaedic implant body of FIG. 10 inserted therein and engaging a fork of the holding device to form the orthopaedic implant.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION OF THE INVENTION

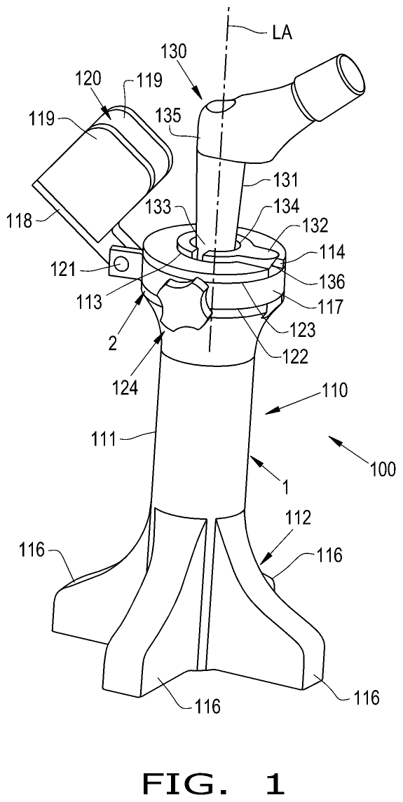

Referring now to the drawings, and more particularly to FIGS. 1-4, an exemplary embodiment of an orthopaedic implant construct 100 provided according to the present invention is illustrated. The construct 100 includes a holding device 110 and an orthopaedic implant 130, illustrated in the form of a femoral component for a hip implant, held by the holding device 110. The holding device 110 may assist in assembling the orthopaedic implant 130, as will be described further herein.

As illustrated, the orthopaedic implant 130 may be a modular orthopaedic implant that includes multiple components, e.g., a body 131 and a sleeve 132, such as a metaphyseal sleeve, coupled to the body 131. The orthopaedic implant 130 includes a stem 133, which may be part of the body 131. The stem 133 can fit inside an opening 134 formed in the sleeve 132 so the stem 133 resides in the opening 134 of the sleeve 132 when the orthopaedic implant 130 is formed. The stem 133 and the opening 134 may define a longitudinal axis LA of the orthopaedic implant 130 that extends in a longest dimension of the orthopaedic implant 130. The body may also include a neck 135 that connects to the stem 133 and extends at an acute angle relative to the longitudinal axis LA. The sleeve 132 may include a protrusion 136 that extends perpendicularly relative to the longitudinal axis LA, i.e., radially with respect to the stem 133, when the stem 133 is inserted in the opening 134. In some embodiments, the stem 133 and the opening 134 each have respective diameters to form a friction fit between the body 131 and the sleeve 132 when the stem 133 is sufficiently forced into the opening 134 and hold the body 131 and the sleeve 132 together. However, the body 131 and the sleeve 132 can be coupled together in other ways, such as using an adhesive or different type of interference fit. In some embodiments, the holding device 110 is configured to be placed in a secondary device, such as a press, that applies a force to the held body 131 and/or the held sleeve 132 to couple the body 131 and the sleeve 132 together and form the orthopaedic implant 130.

The holding device 110 includes a stand 1 with a columnar structure 111 that is coupled to a base 112 and has an opening 113 formed in the structure 111 that is configured to accept a stem, such as the stem 133 of the body 131 of the orthopaedic implant 130 and a groove 114 formed in the structure 111 that is configured to accept a portion of the orthopaedic implant 130, such as the protrusion 136 of the sleeve 132, to rotatably lock the orthopaedic implant 130 in the opening 113. As used herein, the orthopaedic implant 130 is “rotatably locked” in the opening 113 in the sense that at least a portion of the orthopaedic implant 130, such as the sleeve 132, is not freely rotatable within the opening 113, but one or more portions of the orthopaedic implant 130, such as the body 131, may still be rotatable, as will be described further herein.

Many known holding devices for forming orthopaedic implants have several features that make it inconvenient to form the implants. For example, many holding devices are large and take up a lot of sterile space in an operating room where such space is limited. Further, many known holding devices must be held by a healthcare professional, such as a surgical technician, while the implant is being formed, which eliminates the ability of the healthcare professional to perform other tasks. Further, it can be difficult to properly orient the components of a modular implant, such as the body and the sleeve, so the formed orthopaedic implant has components that are angled in the desired manner.

To overcome at least some of the previously described issues, the structure 111 of the holding device 110 is coupled to the base 112, which is configured to stabilize the structure 111 on a surface, such as a surgical table, so the structure 111 stands upright without needing to be held or otherwise supported by a separate structure. In this respect, the base 112 may couple to a bottom of the structure 111 and extend outwardly away from the structure 111 to provide stabilization. For example, the base 112 may include a plurality of feet, illustrated as four feet 116, that each extend away from the structure 111 and are equidistant from one another. By coupling the structure 111 to the base 112 to stabilize the holding device 110 on a surface, the holding device 110 does not need to be held by a healthcare professional to form the orthopaedic implant 130. Further, the stand 1 can include the structure 111 and the base 112 as a relatively compact arrangement, such as a cylindrical structure 111 with the feet 116 extending from the structure 111, so the holding device 110 does not occupy a large amount of space in the operating environment.

The holding device 110 also includes an angle set 2 that includes a ring 117 rotatably coupled to the structure 111. As used herein, the ring 117 is “rotatably coupled” to the structure 111 in the sense that the ring 117 can be rotated about the structure 111 with little, if any, resistance to rotation. It should be appreciated that while the element 117 is described as a “ring” and illustrated as having a circular shape, in some embodiments the ring 117 has a non-circular shape, e.g., an oval or polygonal shape. The ring 117 carries a fork 118 that is configured to engage the orthopaedic implant 130 held by the holding device 110. For example, the fork 118 may engage the neck 135 of the orthopaedic implant 130. The fork 118 engaging the neck 135, i.e., the body 131, of the orthopaedic implant 130 can rotatably lock the body 131 relative to the sleeve 132 (when the protrusion 136 is placed in the groove 114) so the body 131 and the sleeve 132 are rotatably locked together. With the body 131 and the sleeve 132 rotatably locked together, the body 131 can be forced further into the sleeve 132 to form a press fit between the body 131 and the sleeve 132 and secure the components 131, 132 together to form the orthopaedic implant 130.

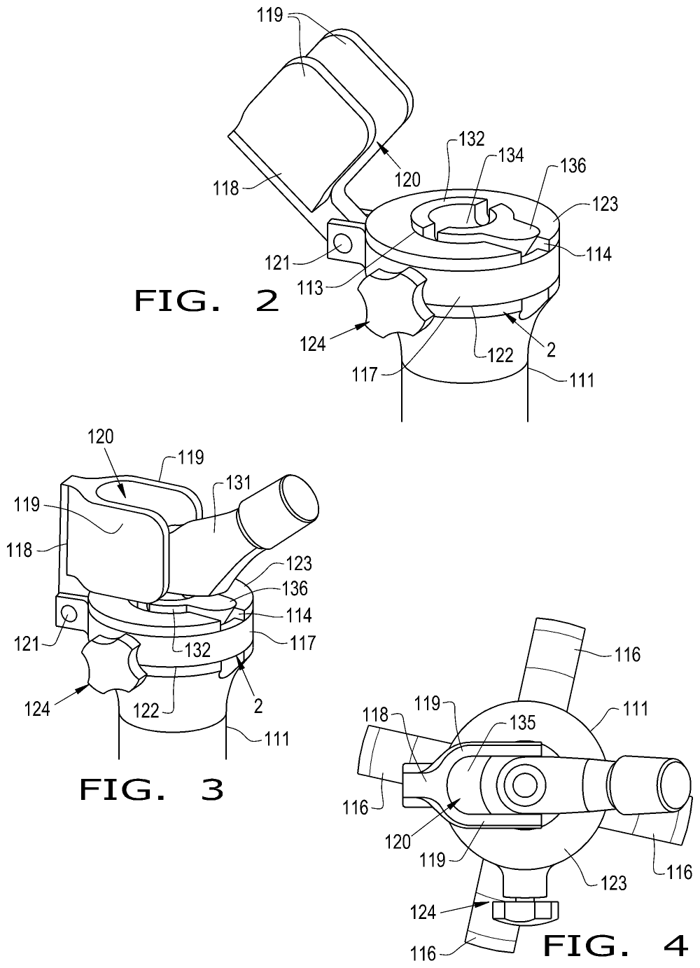

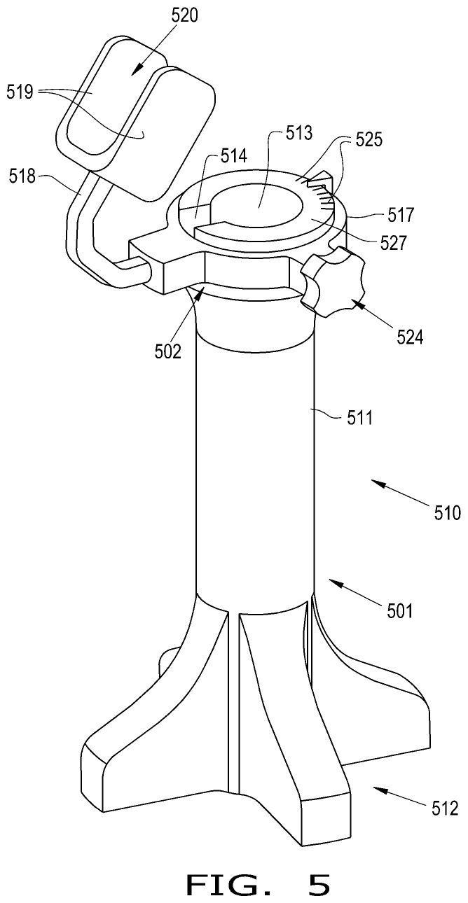

As illustrated, the fork 118 may include a pair of prongs or tines 119 that define an implant slot 120 therebetween, with the fork 118 engaging the orthopaedic implant 130 within the implant slot 120. In some embodiments, there is a small clearance, e.g., 1 mm or less, between the prongs 119 and the orthopaedic implant 130 when the orthopaedic implant 130 is engaged by the fork 118, but the fork 118 still generally prevents appreciable rotation of the engaged portion of the orthopaedic implant 130, i.e., the body 131, relative to the sleeve 132. The fork 118 may be configured to move, relative to the ring 117, between an engaging position where the fork 118 engages the orthopaedic implant 130, as illustrated in FIGS. 3 and 4, and a non-engaging position where the fork 118 is out of engagement with the orthopaedic implant 130, as illustrated in FIGS. 1 and 2. The fork 118 may, for example, be pivotably coupled to the ring 117 by a pivot pin 121 so the fork 118 is configured to pivot between the engaging position and the non-engaging position. In this respect, the fork 118 may move independently of movement of the ring 117 to move between the engaging position and the non-engaging position.

To form the orthopaedic implant 130, the sleeve 132 may initially be placed in the opening 113 so the protrusion 136 resides in the groove 114 and the sleeve 132 is rotatably locked in the opening 113. The ring 117 is positioned so the fork 118 is at a desired angle, relative to the groove 114, such that the body 131 is properly oriented with respect to the sleeve 132 when the body 131 is inserted in the opening 113 and engaged by the fork 118. It should be appreciated that, as used herein, the term “desired” is contextual and should not be interpreted to generally apply to all instances where the holding device provided according to the present invention may be used to assemble orthopaedic implants, i.e., the specific angles and other positions and/or orientations of components that are illustrated and described herein as “desired” are exemplary only. A user may, for example, rotate the ring 117 with respect to the structure 111 so the fork 118 is at the desired position relative to the groove 114 to achieve the desired angle of the body 131 when the body 131 and the sleeve 132 are coupled together in the opening 113. As illustrated in FIG. 2, the body 131 may be partially inserted into the opening 113 and the sleeve 132 while the fork 118 is in the non-engaging position. The fork 118 may be moved, such as pivoted, to the engaging position while the body 131 is partially inserted to engage the body 131, which confirms proper positioning and orientation of the body 131. If the fork 118 is unable to be moved to the engaging position and engage the body 131 when the body 131 is partially inserted in the opening 113 and the sleeve 132, indicating that the body 131 is not in the proper orientation, a user can rotate the body 131 within the opening 113 and the sleeve 132 to an orientation where the fork 118 is able to move to the engaging position and engage the body 131, e.g., with both prongs 119. When the fork 118 moves to the engaging position to engage the body 131 and the body 131 is inserted in the opening 113 and the sleeve 132, the body 131 should be at the set orientation and may be fully inserted into the opening 113 and the sleeve 132 to form a press fit with the sleeve 132 and form the orthopaedic implant 130. It should thus be appreciated that the rotatable ring 117 with the fork 118 can be used to properly orient the body 131 with respect to the sleeve 132 and form the orthopaedic implant 130.

In some embodiments, the structure 111 includes a ring groove 122 and the ring 117 is seated within the ring groove 122. Seating the ring 117 within the ring groove 122 can reduce the risk of the ring 117 uncoupling from the structure 111 during rotation of the ring 117. The ring groove 122 may, for example, be formed adjacent to a rim 123 of the structure 111 so the fork 118 carried by the ring 117 is in close proximity to the body 131 when the body 131 is inserted in the opening 113.

A lock 124 may be provided that is configured to reversibly lock the ring 117 to the structure 111 such that the ring 117 is locked from rotating about the structure 117 when locked to the structure 111. The lock 124 may be, for example, a knob with a threaded portion that is inserted into a threaded opening formed through the ring 117. The threaded portion of the lock 124 may have a sufficient length so that the threaded portion extends through the threaded opening formed through the ring 117 to engage the structure 111. When the threaded portion of the lock 124 engages the structure 111, an interference fit may be formed that rotatably locks the ring 117 to the structure 111. In some embodiments, the structure 111 has locking openings formed in the ring groove 122 of the structure 111 that extend about a circumference of the structure 111 and are sized to accept the threaded portion of the lock 124. In some embodiments, the locking openings formed in the ring groove 122 are threaded, in addition or alternatively to threading the opening formed through the ring 117. If provided, the locking openings formed in the ring groove 122 may be formed in any desired arrangement that allows a user to lock the ring 117 in position where the fork 118 can be used to properly orient the body 131 of the orthopaedic implant 130.

From the foregoing, it should be appreciated that the holding device 110 provided according to the present invention can be used to properly orient components 131, 132 of a modular orthopaedic implant 130 for forming the orthopaedic implant 130. One of the components, such as the sleeve 132, may be rotatably locked to the structure 111 while the other of the components, such as the body 131, may be engaged by the fork 118, which is carried by the ring 117 that is rotatable with respect to the structure 111. When the sleeve 132 is rotatably locked to the structure 111 and the body 131 is engaged by the fork 118, the two components 131, 132 may be properly oriented with respect to one another so the components 131, 132 can be securely coupled together to form the orthopaedic implant 130. For example, the groove 114 and the fork 118 can prevent the sleeve 132 and the body 131, respectively, from rotating relative to one another due to off-axis striking forces as a mallet strikes the body 131 to couple the body 131 and the sleeve 132 together. Since the structure 111 is coupled a base 112 that allows the structure 111 to be stable on a surface, the holding device 110 can be used without needing to be held by a user or other element. In this respect, the holding device 110 provides an easy and convenient way to assemble modular orthopaedic implants 130 from modular components, such as a body 131 and a sleeve 132, in a consistently reproducible way.

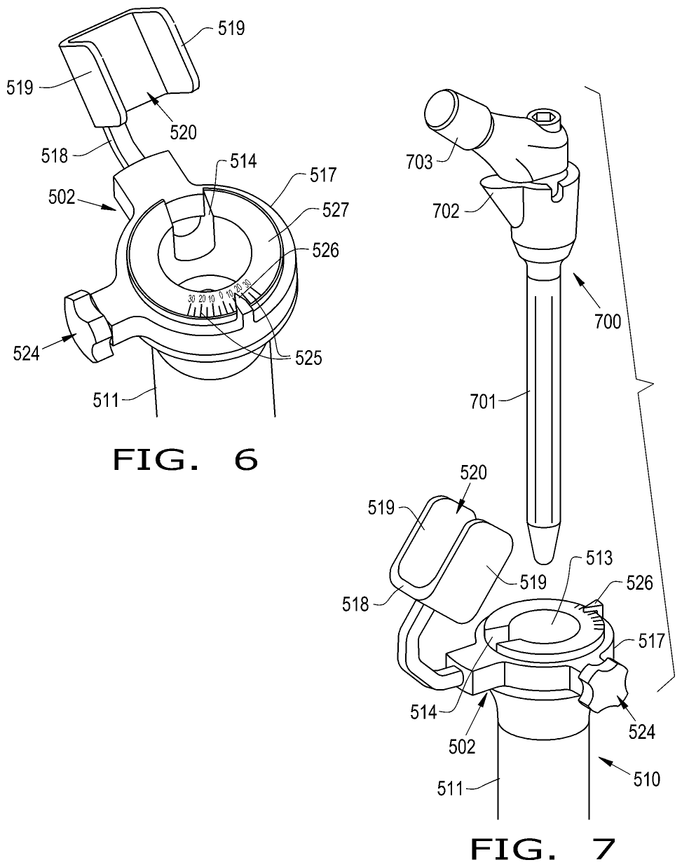

While the previously described holding device 110 includes a fork 118 that is movable between an engaging position and a non-engaging position, in some embodiments a fork is provided that is not movable relative to the ring. Such an embodiment is illustrated in FIGS. 5-11, which illustrate a holding device 510 (illustrated by itself in FIGS. 5-6) with an angle set 502 including a ring 517 that carries a fork 518 that is not movable with respect to the ring 517. The holding device 510, similarly to the holding device 110, includes a stand 501 with a columnar structure 511 that is coupled to a base 512 configured to stabilize the structure 511 on a surface and includes an opening 513 formed in the structure 511 that is configured to accept a stem and a groove 514 formed in the structure 511 that is configured to accept a portion of the orthopaedic implant. The ring 517 is rotatably coupled to the structure 511 and the fork 518 carried by the ring 517, unlike the fork 118, has a fixed orientation with respect to the ring 517 and is not movable, relative to the ring 517, between an engaging position and a non-engaging position. Similarly to the previously described fork 118, the fork 518 can include a pair of prongs 519 that define an implant slot 520 therebetween.

The structure 511 includes a plurality of angle indicators 525 that each correspond to an angle between the groove 514 and the fork 518. When the groove 514 and the fork 518 are aligned, for example, the angle between the groove 514 and the fork 518 may be defined as a zero angle, which represents no rotational offset between the fork 518 and the groove 514. It should be appreciated that the angle indicators 525 of the structure 511 may be accompanied by numerals defining a desired value of the angle between the fork 518 and the groove 514, as illustrated in FIGS. 6 and 8-9. It should be appreciated that the numerals are omitted from illustration in FIGS. 7 and 10-11 only for clarity of illustration. To allow a user to determine what the angle is, the ring 517 may carry an angle marker 526 that is positioned with respect to the fork 518 such that each angle indicator 525 corresponds to the angle between the groove 514 and the fork 518 when the angle marker 526 is aligned with the respective angle indicator 525. As the ring 517 rotates about the structure 511, the fork 518 rotates with respect to the groove 514 and the angle marker 526 correspondingly rotates with respect to the angle indicators 525. In this respect, the angle indicators 525 and the angle marker 526 can be placed on their respective component in any manner that allows the angle between the fork 518 and the groove 514 to be identified. The angle marker 526 may, for example as illustrated, align with the fork 518. It should be appreciated that the angle marker 526 may be offset from the fork 518, i.e., a plane extending through the angle marker 526 does not need to extend through the fork 518, so long as the angle indicators 525 are positioned in an appropriate location of the structure 511 to correspond to the angle between the groove 514 and the fork 518 when the angle marker 526 is aligned with the respective angle indicator 525.

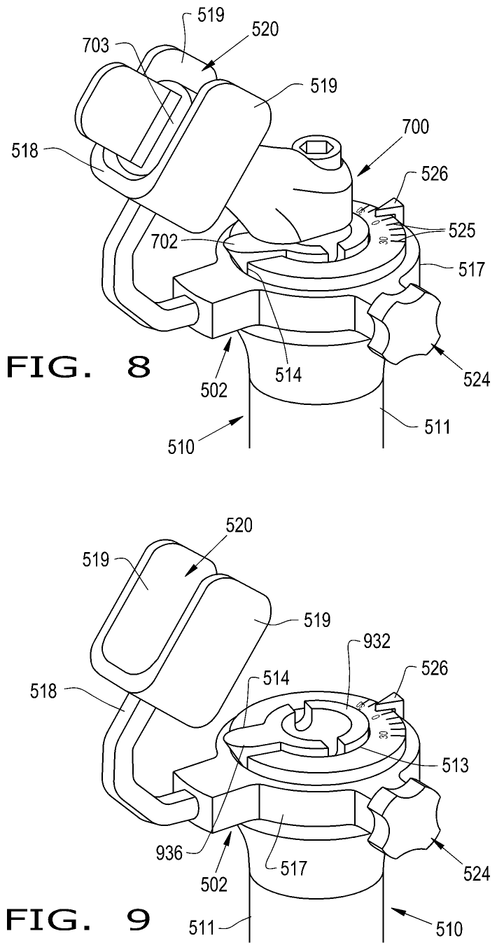

Referring now to FIGS. 7-8, a trial assembly 700 is illustrated being used to prepare the holding device 510 to form an orthopaedic implant construct. The trial assembly 700 includes a stem 701 that is placed in the opening 513 of the structure 511, a protrusion 702 that is placed in the groove 514 of the structure 511, and an alignment neck 703. The alignment neck 703 and the protrusion 702 are oriented with respect to one another in the same manner that it is desired for the corresponding components of an orthopaedic implant to be oriented. The protrusion 702 may be placed in the groove 514 and the ring 517 rotated about the structure 511 until the alignment neck 703 is held in the implant slot 520 to engage the fork 518. When the protrusion 702 is placed in the groove 514 and the fork 518 engages the alignment neck 703, a lock 524 can be used to rotatably lock the ring 517 to the structure 511. A user can visually confirm that the angle between the groove 514 and the fork 518 is the desired value using the angle marker 526 and one of the angle indicators 525, before and/or after the lock 524 rotatably locks the ring 517 to the structure 511. The trial assembly 700 may then be removed from the opening 513 so the holding device 510 can be used to assemble an orthopaedic implant, as will be described further in the context of FIGS. 9-11.

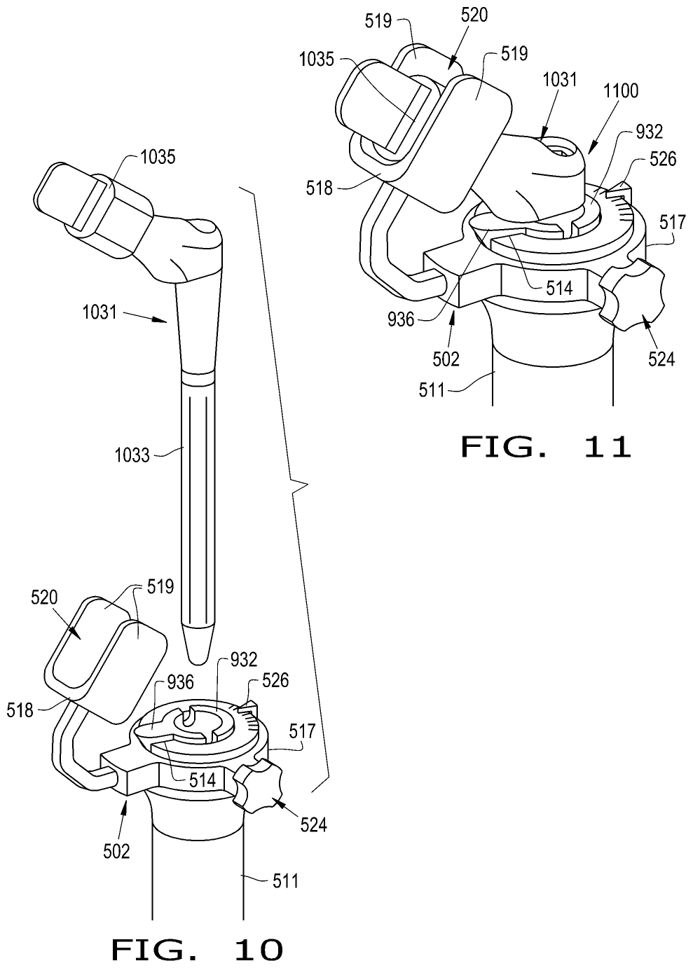

Referring now to FIGS. 9-11, assembly of an orthopaedic implant 1100 (first illustrated fully formed in FIG. 11) using the holding device 510 is illustrated. As illustrated in FIG. 9, which illustrates the holding device 510 after the trial assembly 700 has been removed, the ring 517 and the carried fork 518 are in an orientation so the orthopaedic implant 1100 assembled using the holding device 510 will have the same general orientation of the corresponding components as the trial assembly 700. A sleeve 932 (first illustrated in FIG. 9) of the orthopaedic implant 1100 is inserted in the opening 513 of the structure 511 so a protrusion 936 of the sleeve 932 is placed in the groove t14 to rotatably lock the sleeve 932 to the structure 511. As illustrated in FIG. 10, a body 1031 of the orthopaedic implant 1100 has a stem 1033 that is inserted in the opening 513 of the structure 511 and in the sleeve 932. The body 1031 has a neck 1035 that will be placed in the implant slot 520 of the fork 518 so the fork 518 engages the neck 1035 when the body 1031 is properly oriented with respect to the sleeve 932. As the stem 1033 is inserted in the opening 513 and the sleeve 932, a user may rotate the body 1031 with respect to the sleeve 932, if necessary, until the fork 518 engages the body 1031 while the sleeve 932 is rotatably locked to the structure 511 by the protrusion 936 being placed in the groove 914, which indicates that the body 1031 and the sleeve 932 are properly oriented with respect to one another. A user may then force the body 1031 and the sleeve 932 together to form a friction fit, or otherwise couple the body 1031 and the sleeve 932 together, and form the orthopaedic implant 1100, as illustrated in FIG. 11. The formed orthopaedic implant 110 may then be implanted into a patient and have the same general orientation of corresponding components as the trial assembly 700, which may have been assembled during surgery.

It should be appreciated that, in some embodiments, the sleeve 932 can be placed in the opening 513 so the protrusion 936 is placed in the groove 914 and the stem 1033 is placed partially in the opening 513 and the sleeve 932 so the fork 518 engages the body 1031 in an orientation that is not the desired orientation. The ring 517 can then be rotated to carry the fork 518, which causes rotation of the body 1031 relative to the sleeve 932, to the desired angle, which may be determined using the angle indicators 525 and the angle marker 526. When the body 1031 is at the desired orientation relative to the sleeve 932, the body 1031 can then be forced further into the sleeve 932 and/or otherwise coupled to the sleeve 932 to form the orthopaedic implant 1100. It should thus be appreciated that, according to the present invention, the holding device 510 may be used to form the orthopaedic implant 1100 from the modular components 932, 1031 with or without needing to use a trial assembly 700.

In some embodiments, one or more components of the holding device 510 may be separable from other components to facilitate cleaning and/or sterilization of the holding device 510, which may come into contact with bodily tissues and/or fluids from the trial assembly 700 or otherwise. For example, the structure 511 may be removably coupled to the base 512 so the structure 511 and the base 512 can be separated, e.g., by pulling the structure 511 away from the base 512 until the structure 511 separates from the base 512. Similarly, the ring 517 may be removably coupled to the structure 511 so the ring 517 and the carried fork 518 can be uncoupled from the structure 511. In some embodiments, the structure 511 includes a sleeve cradle 527 that is placed in the opening 513 and has the groove 514 formed therein, with the sleeve cradle 527 being removable from the opening 513 so orthopaedic implants with different shapes, e.g., of the protrusion, can be formed using the holding device 510. It should be appreciated that while one or more of the components of the holding device 510 are previously described as being separable from one another to facilitate cleaning and/or sterilization, the holding device 510 may also be formed as a disposable holding device where having the components separable makes it easier to dispose of the individual components rather than disposing of the entire, assembled holding device 510.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A holding device for assembling orthopaedic implants, the device comprising:
a stand comprising a columnar structure comprising an opening formed in the structure and configured to accept a stem and a groove formed in the structure that is configured to accept a portion of an orthopaedic implant to rotatably lock the orthopaedic implant in the opening and a base coupled to the structure and configured to stabilize the structure on a surface; and
an angle set comprising a ring rotatably coupled to the structure and a fork that is configured to engage the orthopaedic implant and carried by the ring.

2. The holding device of claim 1, wherein the structure comprises a ring groove and the ring is seated within the ring groove.

3. The holding device of claim 2, wherein the ring groove is formed adjacent to a rim of the structure.

4. The holding device of claim 1, wherein the fork is configured to move, relative to the ring, between an engaging position where the fork engages the orthopaedic implant and a non-engaging position where the fork is out of engagement with the orthopaedic implant.

5. The holding device of claim 4, wherein the fork is pivotably coupled to the ring and configured to pivot between the engaging position and the non-engaging position.

6. The holding device of claim 1, wherein the structure defines a cylindrical shape.

7. The holding device of claim 6, wherein the ring defines a circular shape.

8. The holding device of claim 1, wherein the structure comprises a plurality of angle indicators that each correspond to an angle between the groove and the fork.

9. The holding device of claim 8, wherein the ring carries an angle marker that is positioned with respect to the fork such that each angle indicator corresponds to the angle between the groove and the fork when the angle marker is aligned with the respective angle indicator.

10. The holding device of claim 1, further comprising a lock that is configured to reversibly lock the ring to the structure such that the ring is locked from rotating about the structure.

11. An orthopaedic implant construct, comprising:
an orthopaedic implant comprising a stem; and
a holding device holding the orthopaedic implant, the holding device comprising:
a stand comprising a columnar structure comprising an opening formed in the structure and at least partially filled by the stem and a groove formed in the structure and at least partially filled by a portion of the orthopaedic implant so the orthopaedic implant is rotatably locked in the opening and a base coupled to the structure and configured to stabilize the structure on a surface; and
an angle set comprising a ring rotatably coupled to the structure and a fork carried by the ring and engaging the held orthopaedic implant.

12. The orthopaedic implant construct of claim 11, wherein the structure comprises a ring groove and the ring is seated within the ring groove.

13. The orthopaedic implant construct of claim 12, wherein the ring groove is formed adjacent to a rim of the structure.

14. The orthopaedic implant construct of claim 11, wherein the fork is configured to move, relative to the ring, between an engaging position where the fork engages the orthopaedic implant and a non-engaging position where the fork is out of engagement with the orthopaedic implant.

15. The orthopaedic implant construct of claim 14, wherein the fork is pivotably coupled to the ring and configured to pivot between the engaging position and the non-engaging position.

16. The orthopaedic implant construct of claim 11, wherein the structure defines a cylindrical shape.

17. The orthopaedic implant construct of claim 16, wherein the ring defines a circular shape.

18. The orthopaedic implant construct of claim 11, wherein the structure comprises a plurality of angle indicators that each correspond to an angle between the groove and the fork.

19. The orthopaedic implant construct of claim 18, wherein the ring carries an angle marker that is positioned with respect to the fork such that each angle indicator corresponds to the angle between the groove and the fork when the angle marker is aligned with the respective angle indicator.

20. The orthopaedic implant construct of claim 11, further comprising a lock that is configured to reversibly lock the ring to the structure such that the ring is locked from rotating about the structure.
